(19) United States
(12) Patent Application Publication
(10) Pub. No.: US 2007/0059678 A1
(43) Pub. Date: Mar. 15, 2007

(54) Breast milk contaminant detection device

(76) Inventor: Shannon L. King, Lubbock, TX (US)

Correspondence Address: HARSHAW RESEARCH INCORPORATED, P O BOX 418, OTTAWA, KS, 66067, US

(21) Appl. No.: 11/214,290

(22) Filed: Aug. 29, 2005

(51) Int. Cl. C12Q 1/00

(52) U.S. Cl. 435/4

(57) ABSTRACT

A breast milk detection device includes a plurality of test strips slidably and removably coupled to a test housing. Each test strip is connected to an absorbent section so that a breast milk sample deposited thereon is communicated to the test strips. Each test strip includes an analyte-specific moiety for identifying the presence of a predetermined contaminant when contacted by the breast milk sample. The housing includes apertures so that a portion of the test strip may be viewed by a user and the result of the chemical reaction may be analyzed. Indicia indicative of the contaminant being tested for and a legend for interpreting the test results are imprinted upon a top surface of the housing. The breast milk detection device is useful for a nursing mother to determine if her breast milk is safe for the baby.

### BACKGROUND OF THE INVENTION

This invention relates generally to a contaminant detection device and, more particularly, to a device for detecting contaminants in breast milk.

Breast milk is a unique nutritional source that cannot be completely replicated by infant formulas. It is widely regarded that natural breast milk is superior to infant formula with regard to the overall health of the baby and mother and that breast milk provides the most complete source of essential nutrients to an infant. Providing the highest quality nutrients to an infant, of course, is of extreme importance as the infant is still developing systems for fighting germs and other contaminants.

Various proposals for detecting antibodies or bacteria in breast milk or other biological fluids have been disclosed in the art, such as in U.S. Pat. Nos. 4,248,830 and 6,129,680. While assumably effective for their intended purposes, these systems do not provide a convenient and portable home testing device that enables a nursing mother to instantly test a sample of breast milk for the presence of common contaminants.

Therefore, it is desirable to have a breast milk detection device for detecting contaminants in a breast milk sample. Further, it is desirable to have a breast milk detection device that includes an absorbent section for receiving a breast milk sample that is in communication with a plurality of test strips each having a unique chemical composition. In addition, it is desirable to have a breast milk detection device that is easy for a mother to use, read, and interpret.

### SUMMARY OF THE INVENTION

A breast milk contaminant detection device according to the present invention includes a plurality of test strips with each test strip having a chemical composition different from the others and specifically formulated to indicate the presence of a predetermined contaminant. An absorbent section is connected to the test strips for receiving a breast milk sample thereon and communicating it to each test strip. The test strips may be inserted into a housing through openings in a side thereof. Apertures in a top surface of the housing expose portions of the test strips to reveal the test results. The housing top surface further includes indicia indicating what contaminant each test strip is testing for and also includes a legend to aid in user-interpretation of the test results.

The contaminant detection described above requires a basic understanding of human metabolism. A metabolite is any substance produced or used during metabolism (digestion). In the drug context, the term metabolite or analyte refers to the end product that remains after metabolism. These metabolites leach out into the blood over time and are expelled mostly through the urinary tract. In a mother's case, some of the metabolites are expelled through breast milk. With test strips having unique chemical compositions (analytic specific moieties), particular predetermined contaminants may be detected and identified.

Therefore, a general object of the present invention is to provide a breast milk detection device that detects the presence of predetermined contaminants in a sample of breast milk.

Another object of the present invention is to provide a breast milk detection device, as aforesaid, that includes an absorbent section in communication with one or more test strips.

Docket 66508

Still another object of the present invention is to provide a breast milk detection device, as aforesaid, in which each test strip includes a predetermined chemical composition that indicates when the breast milk sample includes a respective contaminant.

Yet another object of the present invention is to provide a breast milk detection device, as aforesaid, that includes a housing having a plurality of openings to reveal a portion of the test strips, the revealed portion indicating the presence of a contaminant or lack thereof.

A further object of the present invention is to provide a breast milk detection device, as aforesaid, in which the housing includes indicia and a legend to aid consumer use of the device.

Other objects and advantages of this invention will become apparent from the following description taken in connection with the accompanying drawings, wherein is set forth by way illustration and example, an embodiment of this invention.

### BRIEF DESCRIPTION OF THE DRAWINGS

FIG. 1 is a perspective view of a breast milk detection device according to a preferred embodiment of the invention;

FIG. 2a is a front view of the device as in FIG. 1;

FIG. 2b is a sectional view of the device taken along line 2b-2b of FIG. 2a;

FIG. 3a is a front view of the detection device housing as in FIG. 1 with the test strip member removed;

FIG. 3b is a perspective view of the housing as in FIG. 3a;

FIG. 3c is an isolated view on an enlarged scale of a portion of the housing as indicated in FIG. 3b; and

FIG. 4 is a perspective view of the test strip member as in FIG. 1 removed from the housing.

### DESCRIPTION OF THE PREFERRED EMBODIMENT

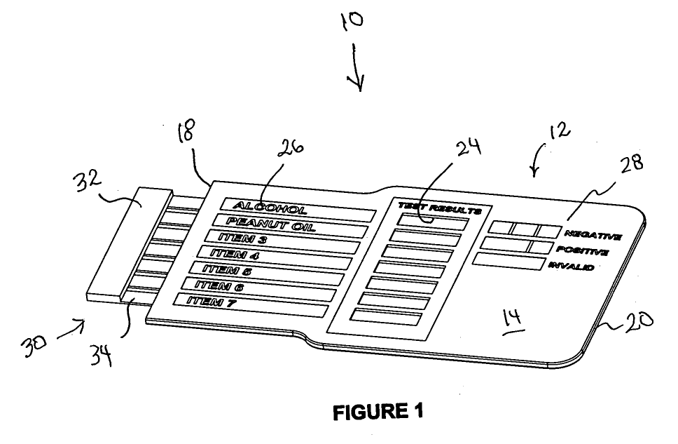

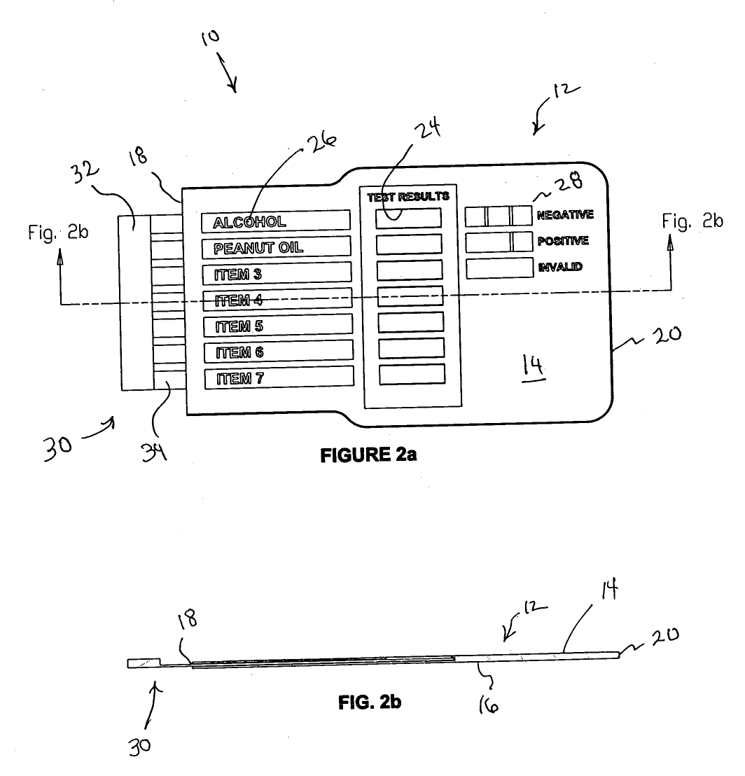

A breast milk contaminant detection device according to the present invention will now be described in detail with reference to FIGS. 1 through 4b of the accompanying drawings. A breast milk detection device 10 according to a preferred embodiment of the present invention includes a housing 12 and a test strip member 30 for insertion into the housing 12.

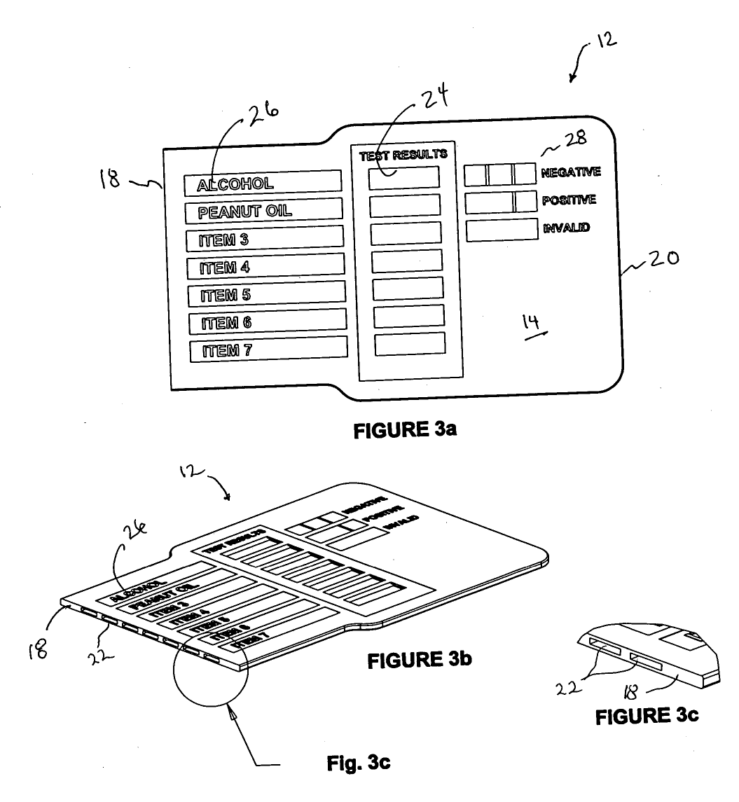

More particularly, the housing 12 includes a top surface 14, a bottom surface 16, and first 18 and second 20 side walls. The top 14 and bottom 16 surfaces are spaced apart such that the housing 12 defines an interior space. The first side wall 18 defines a plurality of spaced apart openings 22 for receiving the test strip member 30 into the interior space as will be further described below (FIGS. 3b and 3c). The openings 22 include configurations complementary to the configuration of corresponding test strips 34. The top surface 14 defines a plurality of apertures 24 spaced apart in vertical alignment so as to expose a portion of respective test strips 34 (FIG. 1). These apertures 24 function as windows for reading test results. Of course, it should be understood that a housing 12 configured for receiving only a single test strip would also be possible.

The top surface 14 of the housing 12 further includes a plurality of indicia 26 imprinted thereon in generally vertical alignment indicative of a particular contaminant (FIG. 1). The indicated contaminant corresponds to the test strip 34 inserted in alignment with a respective indicia. For example, a particular test may include a composition for testing for the presence of alcohol, peanut oil, or other contaminants as will be described in more detail below.

Further, a legend 28 is imprinted on the top surface 14 of the housing 12 for informing a user regarding how to interpret test results viewed through respective apertures 24 (FIG. 1). For example, a single vertical line may indicate a positive test result whereas spaced apart double vertical lines may indicate a negative test result. Other test result indicators and legends, of course, would also be suitable.

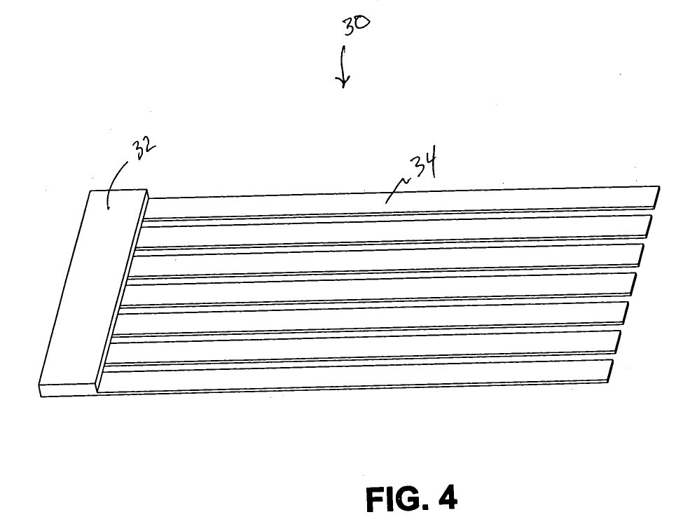

The test strip member 30 includes an absorbent section 32 and a plurality of test strips 34, each test strip having a first end connected to the absorbent section 32 and extending away therefrom (FIG. 4). Therefore, a sample of breast milk may be expressed onto the absorbent section 32 and it is automatically communicated to respective test strips 34. Alternatively, a specimen cup may be used to collect the sample of breast milk and the sample may be delivered to the absorbent section 32 in a separate action.

It is understood that each test strip 34 is generally parallel to the other test strips and spaced apart in a manner complementary to the housing contaminant indicia 26 (FIG. 1). Further, the configuration of the test strips 34 is complementary to the configuration of the first side wall openings 22 of the housing 12 such that the test strips 34 are efficiently received into and removed from the interior space of the housing 12.

Each test strip 34 includes a substrate having a chemical composition different from a chemical composition of any other test strip 34 connected to a test strip member 30. Further, each test strip 34 includes a chemical composition for detecting the presence of a predetermined contaminant when contacted by a sample of breast milk. Chemical compositions suitable for indicating the presence of predetermined contaminants include known enzymes, antibodies, aptamers, and oligomers. These chemical compositions, when appropriately formulated, are capable of indicating the presence of predetermined contaminants such as alcohol, peanut oil, prescription drugs, over-the-counter drugs, illegal drugs, persistent organic pollutants, and other contaminants. Preferably, the absorbent section 32 and substrate of each test strip 34 includes a semi-rigid construction for efficient handling by a user.

With more particular application to the present invention, each test strip 34 includes an analyte specific moiety such as an enzyme, antibody, aptamer, oligomer, or some other recognition element. An analyte present in a breast milk sample reacts with a respective moiety if present in a sufficiently high concentration (a “first” reaction) and then produces a color change or other visible indication (a “second” reaction).

In use, the test strip member 30 may be partially or completed removed from the housing 12 so that a sample of breast milk may be expressed or deposited on the absorbent section 32 thereof. The absorbent section 32 is in direct communication with respective first ends of the test strips 34. As described above, contact between the breast milk sample and the respective analyte moiety causes a chemical reaction which, in turn, causes a second reaction (color or otherwise) indicative of a positive or negative identification result. With the test strip member 30 completely inserted into the housing 12, a user can view the test results through the housing apertures 24 and interpret the results using the legend 28. The test strip member 30 may be replaced by a user for use at a later time. It should be appreciated that a single breast milk sample may result in simultaneous positive identification of multiple contaminants.

It is understood that while certain forms of this invention have been illustrated and described, it is not limited thereto except insofar as such limitations are included in the following claims and allowable functional equivalents thereof.

What is claimed is as follows:

1. A breast milk detection device for use in detecting contaminants in a sample of breast milk, said device comprising:
a plurality of test strips, each test strip having a chemical composition different from a chemical composition of any other test strip and indicative of a predetermined contaminant when contacted by the breast milk sample.

2. The device as in claim 1, wherein each said chemical composition is selected from the group consisting of an enzyme, an antibody, an aptamer, and an oligomer.

3. The device as in claim 1, further comprising an absorbant section situated in communication with each said test strip.

4. The device as in claim 3, wherein each said test strip is generally parallel to every other said test strip.

5. The device as in claim 1, further comprising an absorbant section, and wherein:
each said test strip has a respective first end attached to said absorbant section; and
each said test strip is generally parallel to every other said test strip.

6. The device as in claim 5, wherein:
said absorbant section includes a semi-rigid construction; and
said test strips include a semi-rigid construction.

7. The device as in claim 1, further comprising a housing configured to removably receive said plurality of test strips.

8. The device as in claim 7, wherein said housing defines a plurality of apertures upwardly adjacent said plurality of test strips when said test strips are received in said housing for exposing said test strips when said test strips are received in said housing.

9. The device as in claim 8, wherein said housing includes indicia to identify respective test strips when said test strips are positioned in said housing, each indicium being associated with a respective test strip.

10. The device as in claim 9, wherein said housing includes a legend describing interactions between said test strips and the breast milk sample.

11. The device as in claim 1, further comprising a housing presenting a top surface, a bottom surface, a side, and defining an interior space, said side defining a plurality of openings having configurations complementary to said plurality of test strips for receiving said test strips through said openings into said interior space.

12. The device as in claim 11, wherein said housing top surface defines a plurality of apertures for exposing said test strips when said test strips are received in said housing interior space.

13. The device as in claim 12, further comprising an absorbant section, and wherein:
each said test strip has a respective first end attached to said absorbant section;
each said test strip is generally parallel to every other said test strip;
said housing top surface includes indicia to identify respective test strips when said test strips are received in said interior space; and
said housing top surface includes a legend describing interactions between said test strips and the breast milk sample.

14. The device as in claim 1, wherein said respective test strips change color when said respective chemical compositions react with the predetermined contaminants in the breast milk sample, whereby indicating the presence of the respective predetermined contaminants.

15. The device as in claim 1, wherein said predetermined contaminants are selected from the group consisting of alcohol, prescription drugs, over the counter drugs, illegal drugs, persistent organic pollutants, and allergens.

16. The device as in claim 1, wherein at least one respective test strip chemical composition is indicative of a presence of said respective predetermined contaminant when contacted by the breast milk sample.

17. The device as in claim 1, wherein at least one respective test strip chemical composition is indicative of a quantity of said respective predetermined contaminant when contacted by the breast milk sample.

18. A breast milk detection device for use in detecting contaminants in a sample of breast milk, said device comprising:
a test strip having an analyte-specific moiety indicative of a predetermined contaminant when contacted by the breast milk sample; and
a housing configured to removably receive the test strip.

19. The device as in claim 18, wherein:
said housing presents a top surface, a bottom surface, and a side;
said housing side defines an opening having a configuration complementary to said test strip for receiving said test strip in through said opening into said housing;
said housing top surface defines an aperture for displaying said test strip when said test strip is received in said housing; and
said housing top surface includes a legend describing interactions between said test strip and the sample of breast milk

20. A method of detecting contaminants in a sample of breast milk, said method comprising:
providing a breast milk detection device having a plurality of test strips in communication with an absorbant section, each test strip having a chemical composition different from a chemical composition of any other test strip and indicative of a predetermined contaminant when contacted by the breast milk sample;
introducing the sample of breast milk to the absorbant section;
automatically communicating the sample of breast milk to the plurality of test strips; and
the plurality of test strips indicating respective predetermined contaminants present in the sample of breast milk.
